(19) United States
(12) Patent Application Publication
(10) Pub. No.: US 2015/0313951 A1
(43) Pub. Date: Nov. 5, 2015

(54) FUNCTIONAL FOODS CONTAINING BACILLUS COAGULANS AND NON-DAIRY MILK-LIKE COMPOSITIONS

(71) Applicant: Ganeden Biotech, Inc., Mayfield Heights, OH (US)

(72) Inventors: Howard Cash, Mentor, OH (US); Michael A. Bush, Brecksville, OH (US); Andrew R. Lefkowitz, Mayfield Heights, OH (US); David Keller, Beachwood, OH (US)

(21) Appl. No.: 14/694,481

(22) Filed: Apr. 23, 2015

Related U.S. Application Data
(60) Provisional application No. 61/987,113, filed on May 1, 2014.

(51) Int. Cl. A61K 35/742; A23C 11/10; A61K 9/00

(52) U.S. Cl. CPC A61K 35/742; A61K 9/0095; A23C 11/106; A23C 11/10

(57) ABSTRACT

The present disclosure relates to probiotic compositions and non-dairy milk-like compositions.

### CROSS REFERENCE TO RELATED APPLICATION

This application claims priority under 35 U.S.C. §119(e) to U.S. Provisional Patent Application No. 61/987,113, filed on May 1, 2014, entitled “Functional Foods Containing Bacillus Coagulans and Non-Dairy Milk-Like Compositions,” the disclosure of which is herein incorporated by reference in its entirety.

### FIELD OF THE INVENTION

The present disclosure relates to probiotic compositions.

### BACKGROUND OF THE INVENTION

The gastrointestinal microflora plays a number of vital roles in maintaining gastrointestinal tract function and overall physiological health. The growth and metabolism of the many individual bacterial species inhabiting the gastrointestinal tract depend upon the substrates available to them, most of which are derived from the diet.

Probiotic organisms are non-pathogenic, non-toxigenic microorganisms that are beneficial to the host organism. Since probiotics do not generally permanently colonize the host, they are typically ingested regularly for health promoting properties to persist. Many probiotics are provided in various forms of milk products to benefit the digestive health of individuals. However, the production of non-dairy milk-like products containing probiotics still faces many challenges and therefore there is a great demand to develop these kinds of products to support the growing population in need of them.

### SUMMARY OF THE INVENTION

This disclosure addresses the development non-dairy milk-like compositions containing probiotics. The composition described therein provides probiotic benefit to consumers who are vegans, desire a decreased milk cholesterol content, are lactose intolerance, exhibit allergies towards milk proteins or cannot tolerate or do not wish to consume animal products or by-products.

A dairy product is defined as a food or drink product from the milk of a mammal such as a cow, sheep, or goat. Non-dairy milk-like compositions on the other hand do not contain the milk of a mammal especially cow's milk. A milk-like composition is one that has an appearance and/or texture of cow's milk. In fact, plantmilks have gained popularity by consumers to be used in non-dairy milk-like compositions. Liquids from pressed or pulverized flowers or pressed or pulverized seeds, grains, nuts or legumes comprise non-dairy milk-like compositions. For example, legume based milk is produced from peas, peanuts, lentils, beans or soy. Nutmilk is produced from almonds, cashews, pecans, macadamias, hazelnuts or walnuts. Grainmilk comprises barley, oats, rice or spelt. Lastly, seedmilk is produced from hemp, pumpkin, quinoa, lupines, sesame, pumpkin, sunflower, or coconut.

Particularly, the current invention is based on the discovery that lactic acid-producing bacteria, such as Bacillus coagulans, in non-diary milk compositions supports the immune system as well as the overall health of the digestive system. The microorganisms, e.g., spores remain viable, retain their beneficial probiotic properties, and protect the gut microbiota. Unlike other probiotic bacteria that die in the stomach or small intestine, the probiotic organisms described herein, e.g., Bacillus coagulans generally, or exemplary strains such as GBI-30 or BC³⁰, ATCC Designation Number PTA-6086, germinate in the stomach and/or small intestine. Accordingly, the invention describes probiotic non-dairy milk-like compositions. Specifically, the invention provides an isolated or purified Bacillus coagulans bacterium (e.g., a spore) in non-dairy milk-like compositions.

The compositions described herein are suitable for consumption by a mammal, preferably a human. The compositions are also useful to confer clinical benefit to individuals that are suffering from digestive problems.

The invention provides a composition comprising a non-dairy milk-like composition and an isolated Bacillus coagulans spore (e.g., in an amount of 1×10⁶ to −3×10⁹ colony forming units (CFU) of Bacillus coagulans per serving. For example, the isolated Bacillus coagulans comprise between about 0.000001% to about 50% by weight of the composition, e.g., about 1%, about 10%, about 20%, about 30%, about 40%, or about 50% by weight of the composition. For example, the isolated Bacillus coagulans comprise between about 0.00001% and about 25% by weight of the composition, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the composition. In one example, one unit dose, e.g., one serving, comprises one billion CFU Bacillus coagulans. The size of the serving depends on the nature of the milk product and its consistency. For example, a serving size of non-dairy milk-like milk is 250 mL, kefir is 175 mL, cottage cheese is 116 g, pudding is 125 mL, yoghurt is 175 mL, sour cream is 30 g, cheese is 50 g, ice cream is 125 g, cream cheese is 33 g, coffee creamer is 15 mL and mayonnaise is 15 g.

The invention provides bacterial species including Bacillus coagulans. In a preferred embodiment the strain comprises Bacillus coagulans ATCC Designation Number PTA-6085; GBI-30 or BC³⁰. Examples for strains include Bacillus coagulans hammer Accession No. ATCC 31284, ATCC Numbers: GBI-20, ATCC Designation Number PTA-6085, and GBI-40, ATCC Designation Number PTA-6087 (see U.S. Pat. No. 6,849,256 to Farmer).

In some examples, the isolated Bacillus coagulans is in the form of a spore. Alternatively, the isolated Bacillus coagulans is in the form of a vegetative cell. In another aspect, the isolated Bacillus coagulans is in the form of a mixture of vegetative cells and spores. The Bacillus coagulans in the milk-like composition described herein is predominantly in spore form, e.g., about 75%, about 80%, about 85%, about 90%, about 95%, about 96%, about 97%, about 98%, about 99%, or about 100% spores. Alternatively, the Bacillus coagulans is predominantly in vegetative form, e.g., about 75%, about 80%, about 85%, about 90%, about 95%, about 96%, about 97%, about 98%, about 99%, or about 100% vegetative cells.

Preferably, the composition comprises a non-dairy milk-like product. For example, the non-dairy milk-like product comprises about 1% to about 99% by weight of the composition, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the composition.

In one aspect, the composition comprises a non-dairy milk-like product such as milk, cheese, yoghurt, ice cream, pudding, cream cheese, sour cream, coffee creamer, kefir, cottage cheese or mayonnaise.

Sources for producing non-dairy milk-like compositions, e.g., plantmilks, include legumes, seeds or nuts. For example, legume based milk is produced from peas, peanuts, lentils, beans or soy. Nutmilk is produced from almonds, cashews, pecans, macadamias, hazelnuts or walnuts. Grainmilk is produced from barley, oats, rice or spelt. Seedmilk is produced from hemp, quinoa, lupine, sesame, pumpkin, sunflower, or coconut. Such milks are generally produced by grinding, grating, and/or squeezing the vegetative matter (e.g., leaves or seeds) to extract the liquid therefrom.

Preferably, the isolated Bacillus coagulans is in the form of a spore. Alternatively, the isolated Bacillus coagulans is in the form of a vegetative cell. In another aspect, the isolated Bacillus coagulans is in the form of a mixture of vegetative cells and spores. Preferably, the isolated Bacillus coagulans is in the form of a spore. More preferably, the bacterium is present as at least 80% spores, e.g., at least 85%, at least 90%, at least 95%, at least 98%, or at least 99% spores.

In some cases, the compositions additionally comprise L-alanine and/or inosine to promote the germination of the spores in the stomach and/or small intestine.

A probiotic lactic acid-producing bacteria suitable for use in the methods and compositions produces acid and is non-pathogenic. Purified and/or isolated Bacillus coagulans is particularly useful as a probiotic in the compositions described herein. By “purified” or “substantially purified” is meant a Bacillus coagulans bacterium that is substantially free of contaminating microorganisms or other macromolecules, e.g., polysaccharides, nucleic acids, proteins, or other bacteria. For example, a preparation of B. coagulans is at least 90, 95, 98, 99, 100% w/w B. coagulans.

The Bacillus coagulans hammer strains (GBI-20, ATCC Designation Number PTA-6085; GBI-30 or BC³⁰, ATCC Designation Number PTA-6086; and GBI-40, ATCC Designation Number PTA-6087) of the disclosure are non-pathogenic and generally regarded as safe for use in human nutrition (i.e., GRAS classification) by the U.S. Federal Drug Administration (FDA) and the U.S. Department of Agriculture (USDA), and by those skilled in the art. Furthermore, the Bacillus coagulans hammer strains (GBI-20, ATCC Designation Number PTA-6085; GBI-30 or BC³⁰, ATCC Designation Number PTA-6086; and GBI-40, ATCC Designation Number PTA-6087) germinate at or below human body temperature, rendering them useful as probiotics. Many Bacillus coagulans strains outside the Hammer group have mostly industrial applications, little or no nutritional benefit, and environmental contaminants that have not been evaluated for safety. Moreover, many other non-Hammer strains of Bacillus coagulans grow optimally at temperatures that exceed human body temperature and, thus, do not germinate efficiently in the human body. Such strains are less or not suitable as probiotics for human consumption.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, Genbank/NCBI accession numbers, ATCC numbers and the cell characteristics described in the ATCC listing, and other references mentioned herein are incorporated by reference in their entirety. In the case of conflict, the present specification, including definitions, will control. In addition, the materials, methods, and examples are illustrative only and not intended to be limiting.

### DETAILED DESCRIPTION OF THE INVENTION

There has been an explosion of consumer interest in the active role of food in the well-being and life prolongation, as well as in the prevention of initiation, promotion, and development of chronic diseases. As a result these foods have been called functional foods. Among these, probiotics may exert positive effects on the composition of gut microbiota and overall health. An increased demand in the market of non-dairy probiotics comes from the increased vegetarianism, desire of lower milk cholesterol content, increased lactose intolerance, and allergies towards milk proteins, and the wish to not consume animal products or by-products. All these reasons have made the development of new non-dairy probiotic foods essential.

Lactose-free product are those that lack the presence of lactose. New lactose-free probiotic-containing products have been launched, particularly in fruit-based drinks and cereal. However, application of probiotic cultures in non-dairy products represents a great challenge, because the probiotic viability has to be maintained in the food matrix with the appropriate pH, storage temperature, oxygen levels and absence of competing microorganisms.

The probiotic organisms of the current disclosure are non-pathogenic, non-toxigenic, retain viability during storage, and survive passage through the stomach and small intestine. Non-pathogenic lactic acid-producing bacteria (i.e., “lactic acid bacteria”), such as the exemplary Bacillus coagulans, remain viable and retain their beneficial probiotic properties in non-dairy milk-like compositions. Prior to the disclosure described herein, probiotics were very susceptible to harsh environment of the stomach and small intestine. Unlike other probiotics that die in the stomach or small intestine, the probiotic organisms described herein, e.g., Bacillus coagulans strain GBI-30 or BC³⁰, ATCC Designation Number PTA-6086, germinate in the stomach and/or small intestine. Specifically, the probiotic spores described herein can survive passage through the stomach and small intestine.

### Probiotic Lactic Acid-Producing Bacteria

The non-dairy milk-like compositions include a lactic acid-producing bacterium, such as a spore-forming Bacillus species, e.g., B. coagulans. Preferably, the spore-forming Bacillus species of the disclosure is B. coagulans Hammer. There are many suitable bacteria identified as described herein, although the invention is not limited to currently known bacterial species insofar as the purposes and objectives of the bacteria is described. The property of acid production is important to the effectiveness of the probiotic lactic acid-producing bacteria of this invention.

Exemplary methods and compositions are described herein using Bacillus coagulans as a probiotic. Purified and/or isolated Bacillus coagulans is particularly useful as a probiotic in non-dairy milk-like compositions. Probiotic B. coagulans is non-pathogenic and is generally regarded as safe (i.e., GRAS classification) by the U.S. Federal Drug Administration (FDA) and the U.S. Department of Agriculture (USDA), and by those skilled in the art.

Bacillus coagulans is a non-pathogenic gram positive spore-forming bacteria that produces L(+) lactic acid (dextrorotatory) in fermentation conditions. It has been isolated from natural sources, such as heat-treated soil samples inoculated into nutrient medium (Bergey's Manual off Systemic Bacteriology, Vol. 2, Sneath, P.H.A., et al., eds., Williams & Wilkins, Baltimore, Md., 1986). In addition, Bacillus coagulans ATCC Designation Number PTA-6085; GBI-30 (BC³⁰) is able to survive and colonize the gastrointestinal tract in the bile environment and even grow in this low pH range.

Probiotic Activity of Bacillus coagulans

It is well-documented clinically that many species of bacterial, mycotic and yeast pathogens possess the ability to cause a variety of gastrointestinal disorders including, but not limited to disruption of normal gastrointestinal biochemical function, necrosis of gastrointestinal tissues, and disruption of the bioabsorption of nutrients, and like conditions. The probiotic microorganism-containing compositions described herein inhibit these pathogens. In the present case, subjects are generally healthy, e.g., they have not been diagnosed with a gastrointestinal disease (e.g., irritable bowel syndrome, gastric cancer, constipation, antibiotic-associated diarrhea, or Crohn's disease), an inflammatory bowel condition, or an autoimmune disease. The compositions and methods described herein are useful to maintain digestive health and immune support.

In one aspect, a Bacillus coagulans strain is included in the composition in the form of vegetative cells. In another aspect, the Bacillus coagulans strain is included in the composition in the form of spores.

Specifically, the probiotic organisms described herein, e.g., Bacillus coagulans strain GBI-30 or BC³⁰, ATCC Designation Number PTA-6086, survive passage through the stomach and small intestine.

The probiotic organisms described herein, e.g., Bacillus coagulans strain GBI-30 or BC³⁰, ATCC Designation Number PTA-6086, promote digestive and oral health and support the immune system. The ability of Bacillus coagulans to inhibit various bacterial pathogens was quantitatively ascertained by use of an in vitro assay. This assay is part of a standardized bacterial pathogen screen (developed by the U.S. Food and Drug Administration (FDA)) and is commercially available on solid support disks (DIFCO® BACTROL® Antibiotic Disks). To perform the assay, potato-dextrose plates (DIFCO®) were initially prepared using standard procedures. The plates were then individually inoculated with the bacteria (approximately 1.5×10⁶ CFU) to be tested so as to form a confluent bacterial bed.

Inhibition of microorganisms (e.g. gastrointestinal pathogens) by Bacillus coagulans was subsequently ascertained by placing approximately 1.8×10⁶ CFU of Bacillus coagulans in 10 of broth or buffer, directly in the center of the potato-dextrose plate with one test locus being approximately 8 mm in diameter per plate. A minimum of three test loci were used for each assay. The negative control consisted of a 10 μl volume of a sterile saline solution, whereas the positive control consisted of a 1 μl volume of glutaraldehyde. The plates were then incubated for approximately about 18 hr at 30° C., at which time the zones of inhibition were measured. As designated herein, “excellent inhibition” means the zone was 10 mm or greater in diameter; and “good inhibition” means the zone was greater than 2 mm in diameter but less than 10 mm in diameter.

As expected, no “inhibition” was seen with the negative, saline control, and excellent “inhibition” (approximately 16.2 mm diameter; average of three tests) was seen with the positive, glutaraldehyde control. For the enteric microorganisms tested, the following inhibition by Bacillus coagulans was found: (i) Clostridium species—excellent inhibition; (ii) Escherichia coli—excellent inhibition; (iii) Clostridium species—excellent inhibition, where the zone of inhibition was consistently greater than 15 mm in diameter. Similarly, excellent inhibition was also seen for the opportunistic pathogens Pseudornonas aeruginosa, and Staphylococcus aureus. Pathogenic enteric bacteria which were inhibited by Bacillus coagulans activity include, but are not limited to: Staphylococcus aureus; Staphylococcus epidermidis; Streptococcus pyogenes; Pseudomonas aeruginosa; Escherichia coli (enterohemorragic species); numerous Clostridium species (e.g., Clostridium perfingens, Clostridium botulinum, Clostridium tributrycum, Clostridium sporogenes, and the like); Gardnereia vaginails; Proponbacterium aenes; Aeromonas hydrophia; Aspergillus species; Proteus species; and Klebsiella species.

### Probiotic Non-Dairy Milk-Like Composition

The invention includes a non-dairy milk-like composition, which consists of lactic acid-producing bacteria such as Bacillus coagulans combined with a non-dairy milk-like product. Non-dairy milk-like products are made from plantmilk, which can be derived from grains (barley, oat, rice, spelt), legumes (peas, peanuts, lentils, beans, soy), nuts (almonds, cashews, pecans, macadamias, hazelnuts, walnuts), and seeds (hemp, pumpkin, quinoa, lupines, sesame, pumpkin, sunflower, coconut). Although one thinks primarily of milk as the primary non-dairy milk-like product, additional non-dairy milk-like products include cheese, yoghurt, ice cream, pudding, cream cheese, sour cream, cottage cheese, coffee creamer, kefir and mayonnaise. All these products can be derived from various sources of plantmilk.

An exemplary non-dairy milk-like composition comprises at least about 1×10⁹ viable bacteria. In another aspect, the non-dairy milk-like composition comprises at least about 1×10⁶ to 1×10⁷; at least about 1×10⁷ to 1×10⁸; or at least about 1×10⁸ to 3×10⁹ viable bacteria.

The isolated Bacillus coagulans are at least 85%, at least 90%, at least 95%, or at least 99% pure spores. Alternatively, the isolated Bacillus coagulans is in the form of a vegetative cell. In one aspect, the isolated Bacillus coagulans are at least 85%, at least 90%, or at least 95% pure vegetative cells. In another aspect, the isolated Bacillus coagulans is in the form of a mixture of vegetative cells and spores. The Bacillus coagulans mixture is 90% spores, 10% vegetative cells; 75% spores, 25% vegetative cells; 60% spores, 40% vegetative cells; 50% spores, 50% vegetative cells; 60% vegetative cells, 40% spores; 75% vegetative cells; 25% spores; or 90% vegetative cells, 10% spores.

### EXAMPLE 1

### Preparation of Non-Dairy Milk-Like

Non-dairy milk-like milk can be prepared from plant derived sources such as grains, legumes, nuts, seeds or pulverized plants. A general procedure for the preparation of plantmilk is described below:
a. Soak grains, legumes, nuts, or seeds in water for at least 12 hours. This step softens the selected grain, legume, nut, or seed, which facilitates the upcoming blending process leaving less particles of the plant product in the finished milk. This step may be omitted if a softer plant source is being used.
b. Drain soaked selected grains, legumes, nuts, or seeds and place in blender or other device to disrupt or macerate the plant material.
c. Add water. The ratio of plant source to water may vary, e.g., between 1:2 to 1:4 depending on the preference of the consistency of the milk.
d. Blend the mixture until a smooth consistency of the milk is obtained. An optional step is to strain the resulting composition in order to separate the small particles from the resulting liquid.
e. Add desired amount of spores or vegetative cells and mix with milk to incorporate probiotic component of the product.
f. Refrigerate the probiotic containing milk until further use.

### EXAMPLE 2

### Preparation of Bacillus coagulans Cultures

Bacillus coagulans Hammer bacteria was inoculated and grown to a cell density of about 10⁸ to 10⁹ cells/ml in standard nutrient broth, adjusted to pH 7.0, e.g., using a standard airlift fermentation vessel at 30° C. and standard fermentation methods. The range of MnSO₄ generally acceptable for sporulation is 1 mg/l to 1 g/l. The vegetative cells can actively reproduce up to 45° C., and the spores are stable up to 90° C. After fermentation, the B. coagulans bacterial cells or spores are collected using standard methods (e.g., filtration, centrifugation) and the collected cells and spores can be lyophilized, spray-dried, air-dried or frozen.

A typical yield from the above culture is in the range of about 10⁹ to 10¹⁰ viable spores and more typically about 100 to 150 billion cells/spores per gram before drying. Spores maintain at least 90% viability after drying when stored at room temperature for up to ten years, and thus the effective shelf life of a composition containing B. coagulans spores at room temperature is about 10 years.

### EXAMPLE 3

### Preparation of Bacillus coagulans Spores

A culture of dried B. coagulans spores was prepared as follows. Ten million spores were inoculated into a one liter standard media. The culture was maintained for 72 hours under a high oxygen environment at 37° C. to produce culture having about 150 billion cells per gram of culture. Thereafter, the culture was filtered to remove culture medium liquid, and the bacterial pellet was resuspended in water and freeze-dried. The freeze-dried powder is then ground to a fine powder using standard good manufacturing practice (GMP). A dried composition that comprises at least about 75% (e.g., 80%, 85%, or 90%) spores is then mixed with non-dairy milk-like products.

### OTHER EMBODIMENTS

While the invention has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the disclosure encompassed by the appended claims.

What is claimed:

1. A composition comprising a non-dairy milk-like product and an isolated Bacillus coagulans spore.

2. The composition of claim 1, wherein said isolated Bacillus coagulans is a Bacillus coagulans hammer strain.

3. The composition of claim 1, wherein said isolated Bacillus coagulans is selected from the group consisting of GBI-30 strain (ATCC Designation Number PTA-6086), GBI-20 strain (ATCC Designation Number PTA-6085), and GBI-40 strain (ATCC Designation Number PTA-6087).

4. The composition of claim 1, wherein said composition comprises about 1×10⁶ to 3×10⁹ colony forming units (CFU) of Bacillus coagulans per serving.

5. The composition of claim 1, wherein said composition comprises approximately 1 billion CFU Bacillus coagulans per serving

6. The composition of claim 1, wherein said non-diary milk-like product comprises milk, yoghurt, cheese, ice cream, pudding, cream cheese, sour cream, coffee creamer, kefir, cottage cheese or mayonnaise.

7. The composition of claim 6, wherein said non-diary milk-like product is made from a grain, legume, nut, or seed.

8. The composition of claim 7, wherein said grain comprises barley, oat, rice or spelt.

9. The composition of claim 7, wherein said legume comprises a pea, peanut, lentil, bean or soy legume.

10. The composition of claim 7, wherein said nut comprises an almond, cashew, hazelnut, walnut, macadamia, or pecan nut.

11. The composition of claim 7, wherein seed comprises a hemp, lupine, quinoa, sesame, pumpkin, sunflower, or coconut seed.
